(19) United States
(12) Patent Application Publication
(10) Pub. No.: US 2020/0163829 A1
(43) Pub. Date: May 28, 2020

(54) COLD THERAPY COOLER HAVING A COLOR CHANGING INDICATOR

(71) Applicant: DJO, LLC, Vista, CA (US)

(72) Inventor: Jeremy NATHANSON, Vista, CA (US)

(73) Assignee: DJO, LLC, Vista, CA (US)

(21) Appl. No.: 16/692,541

(22) Filed: Nov. 22, 2019

Related U.S. Application Data
(60) Provisional application No. 62/771,241, filed on Nov. 26, 2018.

(51) Int. Cl. A61H 33/00; A61F 7/10

(52) U.S. Cl. CPC A61H 33/6005; A61F 7/10; A61F 2007/0095; A61H 2203/02; A61H 33/005

(57) ABSTRACT

A cold therapy device is provided. The device includes a fluid tank configured to hold a cooling fluid. The device includes a lid configured to seal an opening in the fluid tank. The device includes a temperature indicator configured to provide an indication of a temperature of the cooling fluid. The device includes a heat-conductive probe. The heat-conductive probe includes a first portion configured to be immersed in the cooling fluid or disposed in direct physical contact with the fluid tank. The heat-conductive probe includes a second portion in physical contact with the temperature indicator and configured to conduct heat between the first portion of the heat-conductive probe and the temperature indicator. A method of using the cold therapy device and a method of manufacturing the cold therapy device are also provided.

### CROSS-REFERENCE TO RELATED APPLICATIONS

This application claims priority to U.S. Provisional Application No. 62/771,241, filed on Nov. 26, 2018, the entire contents of which are hereby incorporated by reference.

### BACKGROUND

### Field of the Disclosure

The present disclosure relates to a cold therapy cooler. Specifically, the apparatuses and methods described herein provide a cold therapy cooler having a thermochromic, color-changing, or otherwise temperature-indicating indicator.

### Description of the Related Technology

Cold therapy is used for treating injuries and controlling inflammation in affected body parts. However, effective cold therapy usually relies on application of a predetermined range of temperatures to the affected body parts, sufficiently cold to appropriately cool the affected body part but not so cold as to cause substantial irritation or damage to the affected body part. One method of providing such a predetermined range of temperatures is using ice baths. However, where such ice baths are maintained within a cooler it can be difficult to determine an instant temperature of the ice bath, whether ice is still present in the bath, and whether ice should be added to the ice bath without opening the cooler and peering inside to visually determine the presence or absence of ice in the bath. In addition, such peering inside generally requires exposing the bath to the ambient environment, which can undesirably accelerate the melting of the ice and increases in the temperature of the bath fluid.

Accordingly, there is a need for solutions that provide the ability to determine a temperature, state or a presence or absence of ice within a cooling fluid for a cold therapy cooler.

### SUMMARY

According to some embodiments, a cold therapy device is provided. The device includes a fluid tank configured to hold a cooling fluid. The device includes a lid configured to seal an opening in the fluid tank. The device includes a temperature indicator configured to provide an indication of a temperature of the cooling fluid. The device includes a heat-conductive probe. The heat-conductive probe includes a first portion configured to be immersed in the cooling fluid or disposed in direct physical contact with the fluid tank. The heat-conductive probe includes a second portion in physical contact with the temperature indicator and configured to conduct heat between the first portion of the heat-conductive probe and the temperature indicator. A method of using the cold therapy device and a method of manufacturing the cold therapy device are also provided.

According to some embodiments, a method of using a cold therapy device is provided. The method includes disposing a cooling fluid in a fluid tank of the cold therapy device. The method includes closing a lid of the cold therapy device to seal an opening in the fluid tank. The method includes immersing a first portion of a heat-conductive probe in the cooling fluid or disposing the first portion of the heat-conductive probe in direct physical contact with the fluid tank. A second portion of the heat-conductive probe is in physical contact with a temperature indicator and is configured to conduct heat between the first portion of the heat-conductive probe and the temperature indicator. A first portion of the temperature indicator is configured to display one of a first color, intensity and/or pattern of light or a second color, intensity and/or pattern of light based on a temperature of the cooling fluid.

According to some embodiments, a method of manufacturing a cold therapy device is provided. The method includes forming a fluid tank configured to hold a cooling fluid. The method includes forming a lid configured to seal an opening in the fluid tank. The method includes disposing at least a first portion of a temperature indicator on the lid or a side of the cold therapy device. The first portion of the temperature indicator is configured to display one of a first color, intensity and/or pattern of light or a second color, intensity and/or pattern of light based on a temperature of the cooling fluid. The method includes disposing a second portion of a heat-conductive probe in physical contact with the temperature indicator. A first portion of the heat-conductive probe is configured to be immersed in the cooling fluid or disposed in direct physical contact with the fluid tank and the second portion of the heat-conductive probe is configured to conduct heat between the first portion of the heat-conductive probe and the temperature indicator.

### BRIEF DESCRIPTION OF THE DRAWINGS

FIG. 1 illustrates a cutaway view of a cold therapy device having a light-modulating indicator, according to some embodiments;

FIG. 2 illustrates a perspective view of the cold therapy device of FIG. 1 wherein the light-modulating indicator displays a first color, according to some embodiments;

FIG. 3 illustrates a perspective view of the cold therapy device of FIG. 1 wherein the light-modulating indicator displays a second color, according to some embodiments;

FIG. 4 illustrates a flowchart of a method of using a cold therapy device having a light-modulating indicator, according to some embodiments;

FIG. 5 illustrates a flowchart of a method of manufacturing a cold therapy device having a light-modulating indicator, according to some embodiments; and

FIG. 6 illustrates a cutaway view of a portion of a cold therapy device having a light-modulating indicator, according to some embodiments

### DETAILED DESCRIPTION

Various aspects of a novel cold therapy device having a color-changing or otherwise light-modulating indicator and associated systems and methods are described more fully hereinafter with reference to the accompanying drawings. However, the disclosure may contemplate many different forms of such devices, indicators and associated methods and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel devices, indicators and associated methods disclosed herein, whether implemented independently of or combined with any other aspect of the disclosure. For example, such devices and indicators may be implemented, or a method may be practiced using any number of the aspects set forth herein. In addition, the scope of the disclosure is intended to cover such devices and indicators or method which is practiced using other structure, functionality, or structure and functionality in addition to or other than the various aspects of the disclosure set forth herein. It should be understood that any aspect disclosed herein may be embodied by one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different applications, some of which are illustrated by way of example in the figures and in the following description of the preferred aspects. The detailed description and drawings are merely illustrative of the disclosure rather than limiting, the scope of the disclosure being defined by the appended claims and equivalents thereof.

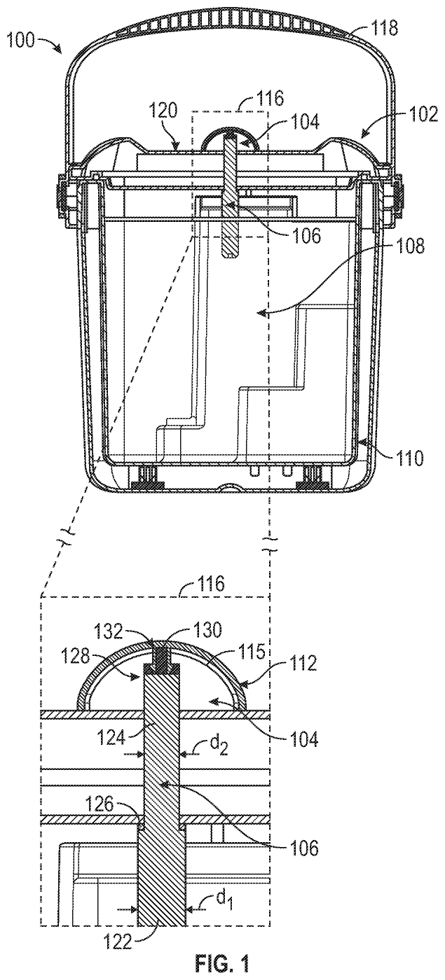

Several aspects of a cold therapy device having a light-modulating indicator will now be described in connection with FIGS. 1-6 below.

FIG. 1 illustrates a cutaway view of a cold therapy device 100 having a light-modulating indicator 104, according to some embodiments. In some embodiments, device 100 comprises a cold therapy cooler. Device 100 comprises a fluid tank 110 configured to hold a cooling fluid 108. In some embodiments, cooling fluid 108 comprises a mixture of ice and liquid water. In some other embodiments, cooling fluid 108 comprises a mixture of ice, liquid water and at least one additive that will decrease the temperature at which cooling fluid 108 freezes to a temperature below the freezing point of pure water.

Device 100 further includes a lid 102 configured to seal an opening in fluid tank 110. In some embodiments, lid 102 is secured to device 100 via one or more hooks, snaps, locks, hinges or any other combination of hinging or securing members. In some embodiments, lid 102 comprises a substantially flat and/or planar portion 120. Lid 102 may be formed of any suitable material, for example plastic or foam. In some embodiments, lid 102 may be formed of a substantially opaque material, which may be less expensive to manufacture compared to a substantially transparent material, such as plexiglass, lexan, or polycarbonate. However, the present disclosure is not so limited and lid 102 may alternatively or additionally be formed in part or in whole of such transparent materials.

Device 100 may further comprise a handle 118. Handle 118 may be secured to an upper portion of device 100 and may be configured to swivel or pivot with respect to device 100, allowing device 100 to be easily picked up and transported. Handle 118 may then be swiveled or pivoted such that handle 118 does not obstruct the range of motion of lid 102.

Device 100 further includes a temperature indicator 104. In some embodiments, temperature indicator 104 has a thermochromic coating 115 configured to change color based on a temperature of cooling fluid 108. In addition, or in the alternative, temperature indicator 104 may comprise an analog or digital temperature indicator, an alcohol thermometer, a mercury thermometer, or any other suitable means for indicating a temperature of cooling fluid 108.

FIG. 1 further illustrates a magnified view 116 of a portion of device 100 including temperature indicator 104 for easy viewing. In some embodiments, temperature indicator 104, and in some embodiments thermochromic coating 115, are substantially dome-shaped, having a convex curvature that bows outward. Such a dome-like and/or convex curvature that bows outward, and in some cases that extends upward away from lid 102, may provide increased visibility of temperature indicator 104 at a distance and from any angle, compared to other shapes, for example, a substantially flat or planar shape. However, the present disclosure is not so limited and at least a portion or an entirety of temperature indicator 104 can alternatively have a substantially flat and/or planar shape. As illustrated in FIG. 1, temperature indicator 104 may be disposed and/or mounted on or at least partially in a top surface of lid 102, which may substantially maximize visibility of temperature indicator 104 from any angle for a user in proximity to device 100 while lid 102 is closed. However, the present disclosure is not so limited and temperature indicator 104 may alternatively be disposed on or at least partially in a side surface of device 100. In such embodiments, the dome-like and/or convex curvature of temperature indicator 104 may bow out to the side of device 100. Utilization of temperature indicator 104 allows a caretaker or patient to readily view whether or not ice should be added to cooling fluid 108 without having to physically opening lid 102 to peer inside device 100. This may be advantageous to nursing staff, for example, making quick rounds to check on patients, and for patients at home who would otherwise have to get up and/or out of bed to check on the status of cooling fluid 108, a serious inconvenience, considering likely post-surgical states of reduced mobility.

Device 100 further comprises a heat-conductive probe 106 having a first portion 122 configured to be immersed in or otherwise in physical contact with a region (e.g., an upper or surface region) of cooling fluid 108, at least when lid 102 is closed, and a second portion 124 coupled to, or in physical contact with, temperature indicator 104. For example, an end of second portion 124 of heat-conductive probe 106 may be physically disposed within a recess 128 in an underside of temperature indicator 104. Second portion 124 is configured to conduct heat between first portion 122 of heat-conductive probe 106 and at least thermochromic coating 115 of temperature indicator 104. In some embodiments not incorporating a thermochromic coating, second portion 124 may be configured to conduct heat between first portion 122 of heat-conductive probe 106 and at least a portion of temperature indicator 104. Second portion 124 of heat-conductive probe 106 conducting heat between first portion 122 and temperature indicator 104, e.g., thermochromic coating 115 of temperature indicator 104, causes a temperature of thermochromic coating 115 and/or of at least a portion of temperature indicator 104 to be substantially the same as, or substantially related to or proportional to, a temperature of cooling fluid 108. Accordingly, heat-conductive probe 106 may comprise a heat-conductive material such as but not limited to metal (e.g., copper, aluminum, steel), a heat pipe, a filled polymer, or any other substantially thermally-conductive material.

Where temperature indicator 104 is disposed and/or mounted on or at least partially in a top surface of lid 102, heat-conductive probe 106 may pass through lid 102. In the alternative, where temperature indicator 104 is disposed and/or mounted on or at least partially in a side surface of device 100, heat-conductive probe 106 may pass through the side of device 100.

As illustrated in magnified view 116 of FIG. 1, first portion 122 of heat-conductive probe 106 may have a first diameter d₁ and second portion 124 of heat-conductive probe 106 may have a second diameter d₂. In some embodiments, first diameter d₁ may be larger than second diameter d₂ (i.e., second diameter d₂ is smaller than first diameter d₁), while a hole in lid 102 or in a side of device 100 through which heat-conductive probe 106 passes may have an intermediate diameter between first diameter d₁ and second diameter d₂ such that heat-conductive probe 106 may be disposed properly within lid 102 or within the side of device 100 but is not able to be inappropriately pulled or pushed completely through lid 102 or the side of device 100. Device 100 may further comprise a seal 126 configured to provide a fluid-tight, and in some cases an air-tight, interface between heat-conductive probe 106 and lid 102 or the side of device 100. In some cases, seal 126 may be configured to be seated against a transition between first portion 122, having first diameter d₁, and second portion 124, having second diameter d₂, the transition thereby providing a surface against which seal 126 may physically press. In some other embodiments, heat-conductive probe 106 may have any other form, for example, having a substantially constant diameter along its length, having the above described diameters of first portion 122 and second portion 124 being reversed, or having one or more fins (not shown) protruding from heat-conductive probe 106 to increase a surface area of heat-conductive probe 106 that is in contact with cooling fluid 108.

In some embodiments, temperature indicator 104 comprises a lens 112 configured to thermally insulate temperature indicator 104 from an ambient environment outside device 100. In some embodiments, lens 112 may be substantially dome-shaped, having a convex curvature that bows outward, similarly to temperature indicator 104. In some embodiments, lens 112 comprise a fastener 130 configured to fasten lens 112 to temperature indicator 104. As shown in magnified view 116 of FIG. 1, fastener 130 may comprise one or more flared tabs configured to snap through and lock into an aperture 132 in temperature indicator 104, thereby securing lens 112 to temperature indicator 104. In some embodiments, aperture 132 may open into recess 128 of temperature indicator 104. In some embodiments, lens 112 may provide an airtight seal between temperature indicator 104 and the external ambient environment, thereby providing thermal isolation to temperature indicator 104. In some embodiments, the space within lens 112 may comprise at least a partial vacuum compared to the external ambient environment. In other embodiments, the space within lens 112 may have a pressure substantially the same as the ambient environment.

In some embodiments, device 100 may be utilized within or as a cold therapy cooler system configured for treating orthopedic injuries and/or for use as a part of a treatment for injury to a body part of a patient. For example, device 100 may be configured to accept, within fluid tank 110, a wrap, compress, brace, cast, water-proof blanket or any other tool or apparatus configured for treatment of an injury to a body part of the patient such that cooling fluid 108 reduces or maintains a temperature of the apparatus substantially at the temperature of cooling fluid 108. In addition, or in the alternative, such a system, comprising device 100, may be configured to supply cooling fluid 108 to such an apparatus when the apparatus is not located within fluid tank 110, for example, utilizing one or more tubes (not shown) coupled between the apparatus and the fluid tank 100 holding cooling fluid 108.

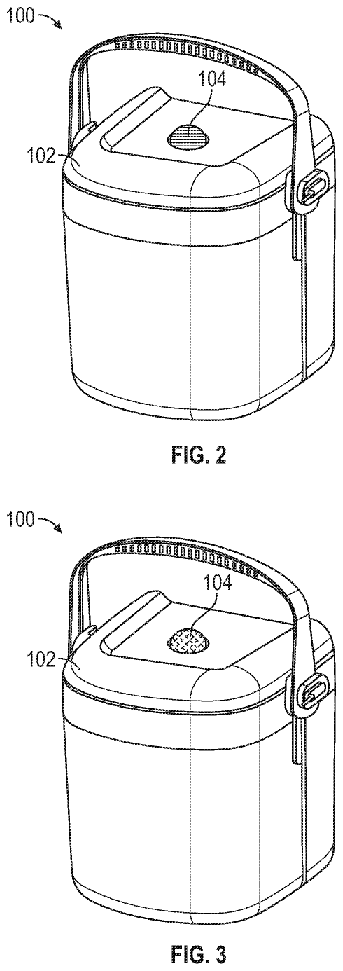

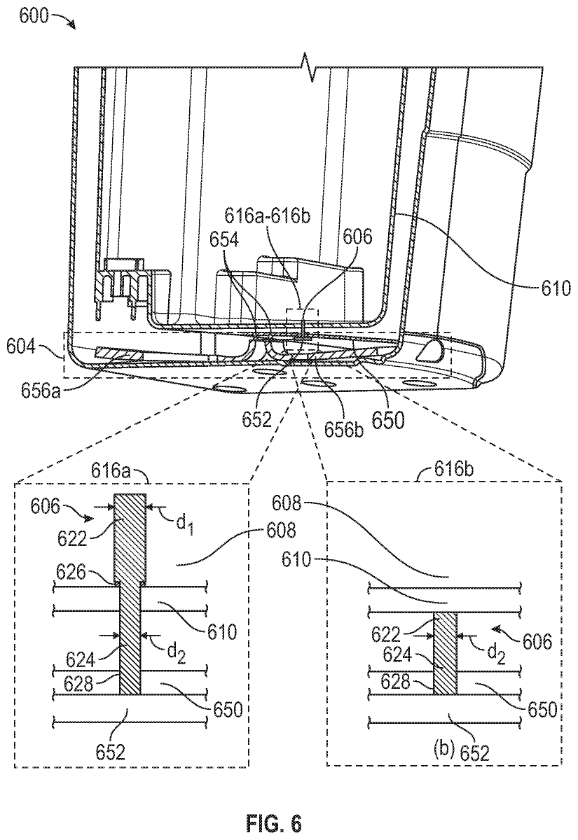

FIG. 2 illustrates a perspective view of cold therapy device 100 and temperature indicator 104 displaying a first color, while FIG. 3 illustrates the same perspective view of cold therapy device 100 and temperature indicator 104 displaying a second color, according to some embodiments. As shown in FIG. 2, in some embodiments, a first color of thermochromic coating 115 of temperature indicator 104, or a first temperature indication provided by at least a portion of temperature indicator 104 itself, indicates a presence of ice in cooling fluid 108 (see FIG. 1), while, as shown in FIG. 3, a second color of thermochromic coating 115 of temperature indicator 104, or a second temperature indication provided by at least a portion of temperature indicator 104 itself, indicates an absence of ice in cooling fluid 108. Accordingly, a presence or absence of ice in fluid tank 110 (see FIG. 1) can be determined without the need for fabricating portions of lid 102, device 100 or fluid tank 110 out of relatively expensive transparent materials or lifting lid 102 to peer inside of device 100, which could undesirably expose cooling fluid 108 to the ambient environment outside device 100, thereby accelerating increases in the temperature of cooling fluid 108. Accordingly, device 100 may be easier to use and may sustain cooling fluid 108 at a suitable temperature for a longer period compared to other designs. Additional and/or alternative embodiments will now be described in connection with FIG. 6.

FIG. 6 illustrates a cutaway view of a portion of a cold therapy device 600 having a light-modulating indicator 604 according to some embodiments. In some embodiments, device 600 comprises a cold therapy cooler. Device 600 comprises a fluid tank 610 configured to hold a cooling fluid 608. In some embodiments, cooling fluid 608 comprises a mixture of ice and liquid water. In some other embodiments, cooling fluid 608 comprises a mixture of ice, liquid water and at least one additive that will decrease the temperature at which cooling fluid 608 freezes to a temperature below the freezing point of pure water.

Device 600 further includes a lid (not shown in FIG. 6 but see lid 102 of device 100 in at least FIG. 1) configured to seal an opening in fluid tank 610. Such a lid may be and/or function substantially as described in connection with FIG. 1, with or without any or all features previously described in connection with at least FIG. 1.

Device 600 may further comprise a handle (not shown in FIG. 6 but see handle 118 of device 100 in at least FIG. 1) secured to an upper portion of device 600 and may be configured to swivel or pivot with respect to device 600, allowing device 600 to be easily picked up and transported. The handle may then be swiveled or pivoted such that the handle does not obstruct the range of motion of the lid.

Device 600 further includes a temperature indicator 604 configured to provide one or more colors, intensities and/or patterns of light based on a temperature of cooling fluid 608. In some embodiments, temperature indicator 604 can additionally or alternatively comprise an analog or digital temperature indicator, an alcohol thermometer, a mercury thermometer, or any other suitable means for indicating a temperature of cooling fluid 608. As illustrated in FIG. 6, at least a portion of temperature indicator 604 can be disposed and/or mounted on or at least partially in a side surface of device 600, which may substantially maximize visibility of that portion of temperature indicator 604 from any angle for a user in proximity to device 600. However, the present disclosure is not so limited and temperature indicator 604 may alternatively be disposed on or at least partially in a top surface of the lid of device 600 (see, e.g., lid 102 and device 100 of FIG. 1) or on or at least partially in a top surface of another portion of the device 600.

Utilization of temperature indicator 604, as will be described in more detail below, allows a caretaker or patient to readily view whether or not ice should be added to cooling fluid 608 without having to physically open lid 602 to peer inside device 600. This may be advantageous to nursing staff, for example, making quick rounds to check on patients, and for patients at home who would otherwise have to get up and/or out of bed to check on the status of cooling fluid 608, a serious inconvenience, considering likely post-surgical states of reduced mobility.

FIG. 6 further illustrates two magnified views 616a, 616b of a portion of device 600 including at least one or more portion(s) of temperature indicator 604 for easy viewing. As illustrated, temperature indicator 604 may comprise a circuit board 650, which may be mounted below and/or to an underside of fluid tank 610. Circuit board 650 may have disposed thereon, and/or therein, a temperature sensor 652 comprising, for example, a thermistor and/or a thermocouple configured to provide an electrical signal indicative of a temperature, and/or temperature condition of cooling fluid 608. The circuit board 650 may further comprise one or more light emitting elements 654. The one or more light emitting elements 654 may comprise light emitting diodes (LEDs), and/or any other suitable light emitting source, configured to emit at least a first color, intensity, and/or pattern of light to indicate a presence of ice in cooling fluid 608 and to emit at least a second color, intensity, and/or pattern of light to indicate an absence of ice in cooling fluid 608. Circuit board 650 may further comprise circuitry configured to control the one or more light emitting elements 654 based on the signal indicative of the measured temperature, or temperature condition, of cooling fluid 608 from temperature sensor 652. In some embodiments, the one or more light emitting elements 654 may be disposed on and/or in any one or more of a bottom surface, a side surface or a top surface of circuit board 650.

Temperature indicator 604 may further comprise one or more light guides 656a, 656b configured to redirect light emitted by the one or more light emitting elements 654 and provide it externally to one or more outer surface(s) on a side and/or top of device 600, thereby allowing a caretaker or patient to readily view the temperature condition of cooling fluid 608, e.g., whether or not ice is present in, and/or whether ice should be added to, cooling fluid 608, without having to physically open the lid to peer inside device 600. In some embodiments, use of the one or more light guides 656a, 656b allows display of the first and/or second color, intensity and/or pattern of light to a user while also allowing the one or more light emitting elements 654, temperature sensor 652 and/or circuit board 650 to be mounted in any desired orientation within device 600. In some embodiments, the one or more light guides 656a,656b may comprise glass, plexiglass, lexan, fiber optic strands or cables, or any other suitable material that is substantially transparent or translucent to the color(s) of light emitted and/or emittable by the one or more light emitting elements 654.

Device 600 may further comprise a heat-conductive probe 606 having a first portion 622 configured to be immersed in, or otherwise in indirect physical contact with, cooling fluid 608 and a second portion 624 coupled to, or otherwise in direct or indirect physical contact with, at least a portion of temperature indicator 604, e.g., temperature sensor 652. For example, an end of second portion 624 of heat-conductive probe 606 may be physically disposed within a recess 628 of circuit board 650, allowing direct and/or indirect physical contact between second portion 624 and temperature sensor 652. However, the present disclosure is not so limited and temperature sensor 652 could alternatively be disposed on an opposite side of circuit board 650, or circuit board 650 could be mounted in a flipped orientation from that illustrated in FIG. 6, such that second portion 624 is not disposed within or through a recess in circuit board 650. Second portion 624 is configured to conduct heat between first portion 622 of heat-conductive probe 606 and at least temperature sensor 652. Second portion 624 of heat-conductive probe 606 conducting heat between first portion 622 and temperature sensor 652 causes a temperature of at least a portion of temperature sensor 652 and/or of second portion 624 of heat-conductive probe 606 to be substantially the same as, or substantially related to or proportional to, a temperature of cooling fluid 608. Accordingly, heat-conductive probe 606 may comprise a heat-conductive material such as but not limited to metal (e.g., copper, aluminum, steel), a heat pipe, a filled polymer, or any other substantially thermally-conductive material.

In some embodiments, as illustrated in the left-most callout 616a, first portion 622 of heat-conductive probe 606 may extend into fluid tank 610, be immersed in cooling fluid 608 and may have a first diameter d₁, while second portion 624 of heat-conductive probe 606 may extend outside and/or through the wall of fluid tank 610 and have a second diameter d₂. In some embodiments, first diameter d₁ may be larger than second diameter d₂ (i.e., second diameter d₂ is smaller than first diameter d₁), while a hole in a bottom or side of fluid tank 610, through which heat-conductive probe 606 passes may have an intermediate diameter between first diameter d₁ and second diameter d₂ such that heat-conductive probe 606 may be disposed properly within fluid tank 610 but is not able to be inappropriately pulled or pushed completely through fluid tank 610. In such embodiments, device 600 may further comprise a seal 626 configured to provide a fluid-tight, and in some cases an air-tight, interface between heat-conductive probe 606 and fluid tank 610. In some cases, seal 626 may be configured to be seated against a transition between first portion 622, having first diameter d₁, and second portion 624, having second diameter d₂, the transition thereby providing a surface against which seal 626 may physically press. In some other embodiments, heat-conductive probe 606 may have any other form, for example, having a substantially constant diameter along its length, having the above-described diameters of first portion 622 and second portion 624 being reversed, or having one or more fins (not shown) protruding from heat-conductive probe 606 to increase a surface area of heat-conductive probe 606 in contact with cooling fluid 608.

In some embodiments, as illustrated in the right-most callout 616b, first portion 622 of heat-conductive probe 606 may not be immersed in cooling fluid 608 and may, instead be physically disposed against a bottom or side surface of fluid tank 610. First portion 622 and second portion 624 may each have a diameter d₂, e.g., heat-conductive probe 606 may have a substantially constant diameter along its length. However, the present disclosure is not so limited and first portion 622 may have a first diameter d₁, while second portion 624 may have a second diameter d₂, as described above. In embodiments corresponding to callout 616b, heat-conductive probe 606 may not extend through a wall of fluid tank 610. In such embodiments, seal 626 may not be utilized.

In some embodiments, device 600 may be utilized within or as a cold therapy cooler system configured for treating orthopedic injuries and/or for use as a part of a treatment for injury to a body part of a patient. For example, device 600 may be configured to accept, within fluid tank 610, a wrap, compress, brace, cast, water-proof blanket or any other tool or apparatus configured for treatment of an injury to a body part of the patient such that cooling fluid 608 reduces or maintains a temperature of the apparatus substantially at the temperature of cooling fluid 608. In addition, or in the alternative, such a system, comprising device 600, may be configured to supply cooling fluid 608 to such an apparatus when the apparatus is not located within fluid tank 610, for example, utilizing one or more tubes (not shown) coupled between the apparatus and the fluid tank 600 holding cooling fluid 608.

Accordingly, a presence or absence of ice in fluid tank 610 can be determined without the need for fabricating portions of the lid, device 600 or fluid tank 610 out of relatively expensive transparent materials or lifting the lid to peer inside of device 600, which could undesirably expose cooling fluid 608 to the ambient environment outside device 600, thereby accelerating increases in the temperature of cooling fluid 608. Accordingly, device 600 may be easier to use and may sustain cooling fluid 608 at a suitable temperature for a longer period compared to other designs.

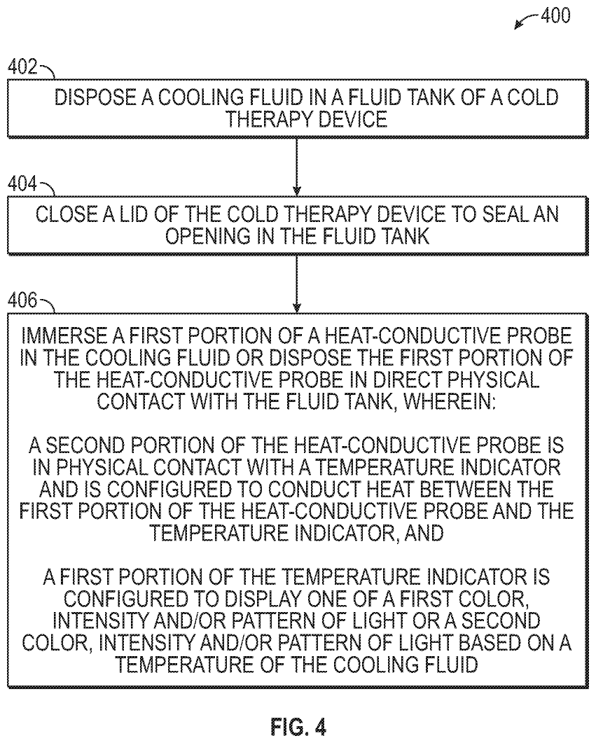

FIG. 4 illustrates a flowchart 400 of a method for using a cold therapy device having a color-changing indicator, according to some embodiments. In some embodiments, flowchart 400 may be utilized for a system, comprising device 100 of FIGS. 1-3 and/or device 600 of FIG. 6, configured for treating orthopedic injuries and/or for use as a part of a treatment for injury to a body part of a patient, as previously described. Flowchart 400 will now be described in connection with cold therapy device(s) 100, 600 as previously described in connection with FIGS. 1-3 and 6.

Block 402 includes disposing a cooling fluid in a fluid tank of the cold therapy device. For example, as previously described in connection with FIGS. 1-3 and FIG. 6, cooling fluid 108,608 may be disposed in fluid tank 110,610 of cold therapy device 100,600. In some embodiments, cooling fluid 108,600 comprises a mixture of water and ice.

Block 404 includes closing a lid of the cold therapy device to seal an opening in the fluid tank. For example, as previously described in connection with FIGS. 1-3 and FIG. 6, lid 102 of cold therapy device 100,600 is configured to seal an opening in fluid tank 110,610 and/or in cold therapy device 100,600.

Block 406 includes immersing a first portion of a heat-conductive probe in the cooling fluid or disposing the first portion of the heat-conductive probe in direct physical contact with the fluid tank, wherein: a second portion of the heat-conductive probe is in physical contact with a temperature indicator and is configured to conduct heat between the first portion of the heat-conductive probe and the temperature indicator, and a first portion of the temperature indicator is configured to display one of a first color, intensity and/or pattern of light or a second color, intensity and/or pattern of light based on a temperature of the cooling fluid.

For example, as previously described in connection with FIGS. 1-3, where temperature indicator 104 is disposed in lid 102, closing lid 102 when fluid tank 110 is sufficiently filled with cooling fluid 108 will cause first portion 122 of heat-conductive probe 106 to be immersed in cooling fluid 108. Alternatively, where temperature indicator 104 is disposed in or on a side of cold therapy device 100, sufficiently filing fluid tank 110 with cooling fluid 108 will cause first portion 122 of heat-conductive probe 106 to be immersed in cooling fluid 108 (e.g., in an upper or surface region of cooling fluid 108). Second portion 124 of heat-conductive probe 106 is in physical contact with temperature indicator 104, which in some embodiments, comprises thermochromic coating 115 configured to display one of a first color, intensity and/or pattern of light or a second color, intensity and/or pattern of light based on a temperature of cooling fluid 108. In such embodiments, second portion 124 of heat-conductive probe 106 is configured to conduct heat between first portion 122 of heat-conductive probe 106 and at least thermochromic coating 115 of temperature indicator 104. In some embodiments, temperature indicator 104 may be configured to provide one or more indications (e.g., color indications, graphical indications, numeric indications, or any other suitable indications) based on and/or indicative of temperatures of cooling fluid 108.

In an additional and/or alternative example, as previously described in connection with FIG. 6, where at least a portion of temperature indicator 604 is disposed at a side of cold therapy device 600 and first portion 622 of heat-conductive probe 606 is configured to be immersed in cooling fluid 608 (e.g., at least callout 616a of FIG. 6), sufficiently filing fluid tank 610 with cooling fluid 608 will cause first portion 622 of heat-conductive probe 106 to be immersed in cooling fluid 608 (e.g., in a lower region of cooling fluid 608). Alternatively, first portion 622 of heat-conductive probe 606 may be configured to be disposed in direct physical contact with fluid tank 610 as described in connection with, e.g., at least callout 616b of FIG. 6. Second portion 624 of heat-conductive probe 606 is in physical contact with temperature indicator 604, e.g., temperature sensor 652, and is configured to conduct heat between first portion 622 of heat-conductive probe 606 and temperature sensor 652. In such embodiments, a first portion of temperature indicator 604, e.g., one or more of light guides 656a, 656b, is configured to display one of a first color, intensity and/or pattern of light or a second color, intensity and/or pattern of light based on a temperature of cooling fluid 608.

In some embodiments, temperature indicator 604 comprises temperature sensor 652 configured to generate an electrical signal indicative of the temperature of cooling fluid 608, one or more light emitting elements 654 configured to generate the first or second color, intensity and/or pattern of light, and circuitry configured to control the one or more light emitting elements 654 based on the electrical signal. In such embodiments, the first portion of temperature indicator 604 may comprise at least one light guide 656a,656b configured to provide the first or second color, intensity and/or pattern of light externally of cold therapy device 600. In such embodiments, the method described by flowchart 400 may additionally include determining one of a presence of ice in cooling fluid 608 based on the at least one light guide 656a, 656b displaying the first color, intensity and/or pattern of light, or an absence of ice in cooling fluid 608 based on at least one light guide 656a, 656b displaying the second color, intensity and/or pattern of light.

In some embodiments, the method described by flowchart 400 may additionally include causing a temperature of at least a portion of temperature sensor 652 to substantially approach a same temperature as cooling fluid 618 when first portion 622 of heat-conductive probe 606 is immersed in cooling fluid 608 or disposed in direct physical contact with fluid tank 610.

In some embodiments, the first portion of temperature indicator 104 may comprise thermochromic coating 115 and the method described by flowchart 400 may additionally include determining one of a presence of ice in cooling fluid 108 based on thermochromic coating 115 displaying the first color (e.g., indicating a temperature substantially at or below the melting point of water ice, 0 degrees Celsius), or an absence of ice in cooling fluid 108 based on thermochromic coating 115 displaying the second color (e.g., indicating a temperature substantially above the melting point of water ice, greater than 0 degrees Celsius).

In some embodiments, such determining may be carried out by visual inspection of thermochromic coating 115 of temperature indicator 104 displaying either the first color or the second color through lens 112 covering temperature indicator 104. In some other embodiments, such determining may be carried out by visual inspection of temperature indicator 104 displaying either the first temperature indication or the second temperature indication through lens 112 covering temperature indicator 104. Lens 112 is configured to thermally insulate temperature indicator 104 from an ambient environment outside cold therapy device 100.

In some embodiments, the method described by flowchart 400 may further include causing a temperature of the thermochromic coating, and/or at least a portion of the temperature indicator, to substantially approach a same temperature as the cooling fluid when the first portion of the heat-conductive probe is immersed in the cooling fluid. For example, as previously described in connection with FIGS. 1-3, second portion 124 of heat-conducting probe 106 being configured to conduct heat from first portion 122 of heat-conducting probe 106 to thermochromic coating 115 of temperature indicator 104 will cause a temperature of thermochromic coating 115 to substantially approach a same temperature as cooling fluid 108 when first portion 122 of heat-conductive probe 106 is immersed in cooling fluid 108. As previously described, in some embodiments, second portion 124 of heat-conducting probe 106 may be configured to conduct heat from first portion 122 of heat-conducting probe 106 to at least a portion of temperature indicator 104, which will cause a temperature of at least a portion of temperature indicator 104 to approach a same temperature as cooling fluid 108 when first portion 122 of heat-conductive probe 106 is immersed in cooling fluid 108.

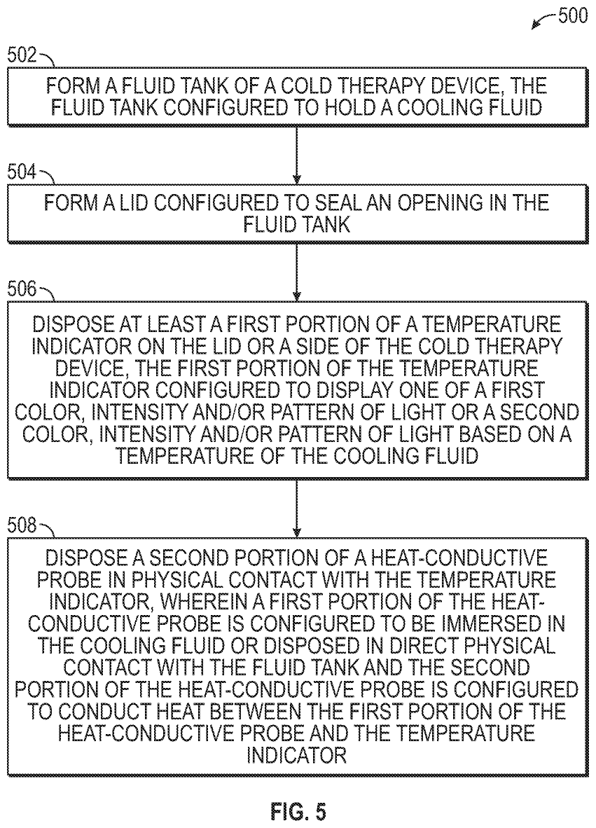

FIG. 5 illustrates a flowchart 500 of a method of manufacturing a cold therapy device having a color-changing indicator, according to some embodiments. In some embodiments, cold therapy device 100 may be a part of a system configured for treating orthopedic injuries and/or for use as a part of a treatment for injury to a body part of a patient, as previously described. Flowchart 500 will now be described in connection with cold therapy device 100 as previously described in connection with FIGS. 1-3 and/or cold therapy device 600 as previously described in connection with FIG. 6.

Block 502 includes forming a fluid tank configured to hold a cooling fluid. For example, as previously described in connection with FIGS. 1-3 and FIG. 6, fluid tank 110,610 may be formed as illustrated such that it is configured to hold cooling fluid 108,608.

Block 504 includes forming a lid configured to seal an opening in the fluid tank. For example, as previously described in connection with FIGS. 1-3 and FIG. 6, lid 102 may be formed as illustrated in FIGS. 1-3 such that it is configured to seal an opening in fluid tank 110,610.

Block 506 includes disposing at least a first portion of a temperature indicator on at least one of the lid and a side of the cold therapy device. For example, as previously described in connection with FIGS. 1-3, temperature indicator 104 may be disposed on lid 102 or at a side of fluid tank 110, which may ultimately be on an outer side of cold therapy device 100. In some embodiments, the first portion of temperature indicator 104 comprises thermochromic coating 115, which may be substantially dome-shaped, and which may be disposed on lid 110. In such embodiments, thermochromic coating 115 may have a convex curvature that bows outward from cold therapy device 100. In some other embodiments, temperature indicator 104 may provide a first temperature indication when a temperature of cooling fluid 108 is such that ice is still present in cooling fluid 108 (e.g., a temperature substantially at or below the melting point of water ice, 0 degrees Celsius), and may provide a second temperature indication when a temperature of cooling fluid 108 is such that ice is not still present in cooling fluid 108 (e.g., a temperature substantially above the melting point of water ice, greater than 0 degrees Celsius).

As yet another example, as previously described in connection with FIG. 6, the first portion of temperature indicator 604 may include at least one light guide 656a,656b disposed at the side of cold therapy device 600 and configured to provide the first or second color, intensity and/or pattern of light externally of cold therapy device 600.

Block 508 includes disposing a second portion of a heat-conductive probe in physical contact with the temperature indicator, wherein a first portion of the heat-conductive probe is configured to be immersed in the cooling fluid or disposed in direct physical contact with the fluid tank. In some embodiments, the second portion of the heat-conductive probe is configured to conduct heat between the first portion of the heat-conductive probe and the temperature indicator.

For example, as previously described in connection with FIGS. 1-3, second portion 124 of heat-conductive probe 106 may be brought into physical contact with temperature indicator 104. First portion 122 of heat-conductive probe 106 is configured to be immersed in cooling fluid 108 and second portion 124 of heat-conductive probe 106 is configured to conduct heat between first portion 122 and at least thermochromic coating 115 of temperature indicator 104, or alternatively at least a portion of temperature indicator 104. In some embodiments, second portion 124 of heat-conductive probe 106 is at least partially physically disposed within recess 128 in temperature indicator 104. In some such embodiments, temperature indicator 104 can be substantially dome-shaped, comprising a convex curvature that bows outward from cold therapy device 100. In addition, in some embodiments, first portion 122 of heat-conductive probe 106 may have a first diameter d₁ and second portion 124 of heat-conductive probe 106 may have a second diameter d₂ smaller than first diameter d₁.

As another example, as previously described in connection with FIG. 6, second portion 624 of heat-conductive probe 606 may be brought into physical contact with temperature sensor 652 of temperature indicator 604. First portion 622 of heat-conductive probe 606 is configured to be immersed in cooling fluid 608 or disposed in direct physical contact with fluid tank 610 and second portion 624 of heat-conductive probe 606 is configured to conduct heat between first portion 622 and temperature sensor 652 of temperature indicator 604. In some embodiments, second portion 624 of heat-conductive probe 606 is at least partially physically disposed within recess 628 in circuit board 650 of temperature indicator 604. In addition, in some embodiments, first portion 622 of heat-conductive probe 606 may have a first diameter d₁ and second portion 624 of heat-conductive probe 606 may have a second diameter d₂ smaller than first diameter d₁.

In some embodiments, as those described in connection with FIG. 6, the method described by flowchart 500 may further include mounting circuit board 650 on or adjacent to fluid tank 610. In such embodiments, circuit board 650 may form a part of temperature indicator 604. Circuit board 500 can include temperature sensor 652 configured to generate an electrical signal indicative of the temperature of cooling fluid 608, one or more light emitting elements 654 configured to generate the first and/or second color, intensity and/or pattern of light, and circuitry configured to control the one or more light emitting elements 654 based on the electrical signal.

In some embodiments, the method described by flowchart 500 may further include disposing lens 112 over temperature indicator 104, lens 112 being configured to thermally insulate temperature indicator 104 from an ambient environment outside cold therapy device 100.

Various modifications to the implementations described in this disclosure can be readily apparent to those skilled in the art, and any generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that may be described in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

In addition, the methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

While the foregoing is directed to aspects of the present disclosure, other and further aspects of the disclosure may be devised without departing from the basic scope thereof, and the scope thereof is determined by the claims that follow.

What is claimed is:

1. A cold therapy device, comprising:
a fluid tank configured to hold a cooling fluid;
a lid configured to seal an opening in the fluid tank;
a temperature indicator configured to provide an indication of a temperature of the cooling fluid; and
a heat-conductive probe having:
a first portion configured to be immersed in the cooling fluid or disposed in direct physical contact with the fluid tank; and
a second portion in physical contact with the temperature indicator and configured to conduct heat between the first portion of the heat-conductive probe and the temperature indicator.

2. The cold therapy device of claim 1, wherein at least a first portion of the temperature indicator is mounted to a side of the cold therapy device.

3. The cold therapy device of claim 2, wherein the first portion of the temperature indicator comprises at least one light guide configured to provide the indication of the temperature of the cooling fluid, and wherein the temperature indicator comprises:
a circuit board comprising:
a temperature sensor configured to generate an electrical signal indicative of the temperature of the cooling fluid,
one or more light emitting elements configured to generate the indication of the temperature of the cooling fluid, and
circuitry configured to control the one or more light emitting elements based on the electrical signal from the temperature sensor.

4. The cold therapy device of claim 3, wherein the indication of the temperature of the cooling fluid comprises one of:
a first color, intensity, and/or pattern of light indicating a presence of ice in the cooling fluid; and
a second color, intensity, and/or pattern of light indicating an absence of ice in the cooling fluid.

5. The cold therapy device of claim 1, wherein the first portion of the heat-conductive probe has a first diameter and the second portion of the heat-conductive probe has a second diameter smaller than the first diameter.

6. The cold therapy device of claim 1, wherein the second portion of the heat-conductive probe is at least partially physically disposed within a recess within a portion of the temperature indicator.

7. The cold therapy device of claim 1, wherein the heat-conductive probe comprises at least one fin configured to at least partially contact the cooling fluid.

8. The cold therapy device of claim 1, wherein at least a first portion of the temperature indicator is mounted to the lid such that the first portion of the temperature indicator is visible to a user in proximity to the cold therapy device with the lid closed.

9. The cold therapy device of claim 8, wherein the first portion of the temperature indicator comprises a thermochromic coating configured to change color based on the temperature of the cooling fluid and wherein the second portion of the heat-conductive probe is configured to conduct heat between the first portion of the heat-conductive probe and the thermochromic coating.

10. The cold therapy device of claim 8, wherein the first portion of the temperature indicator is substantially dome-shaped, comprising a convex curvature that bows outward from the cold therapy device.

11. The cold therapy device of claim 9, wherein the second portion of the heat-conductive probe conducting heat between the first portion of the heat-conductive probe and the thermochromic coating causes a temperature of the thermochromic coating to be substantially the same as a temperature of the cooling fluid.

12. The cold therapy device of claim 9, wherein a first color of the thermochromic coating indicates a presence of ice in the cooling fluid and a second color of the thermochromic coating indicates an absence of ice in the cooling fluid.

13. The cold therapy device of claim 9, wherein the temperature indicator comprises a lens configured to thermally insulate the temperature indicator from an ambient environment outside the cold therapy device.

14. A method of using a cold therapy device, the method comprising:
disposing a cooling fluid in a fluid tank of the cold therapy device;
closing a lid of the cold therapy device to seal an opening in the fluid tank; and
immersing a first portion of a heat-conductive probe in the cooling fluid or disposing the first portion of the heat-conductive probe in direct physical contact with the fluid tank, wherein:
a second portion of the heat-conductive probe is in physical contact with a temperature indicator and is configured to conduct heat between the first portion of the heat-conductive probe and the temperature indicator, and
a first portion of the temperature indicator is configured to display one of a first color, intensity and/or pattern of light or a second color, intensity and/or pattern of light based on a temperature of the cooling fluid.

15. The method of claim 14, wherein:
the temperature indicator comprises:
a temperature sensor configured to generate an electrical signal indicative of the temperature of the cooling fluid,
one or more light emitting elements configured to generate the first or second color, intensity and/or pattern of light, and
circuitry configured to control the one or more light emitting elements based on the electrical signal;
the first portion of the temperature indicator comprises at least one light guide configured to provide the first or second color, intensity and/or pattern of light externally of the cold therapy device; and
the method comprises determining one of:
a presence of ice in the cooling fluid based on the at least one light guide providing the first color, intensity and/or pattern of light; or
an absence of ice in the cooling fluid based on at least one light guide providing the second color, intensity and/or pattern of light.

16. The method of claim 15, comprising causing a temperature of at least a portion of the temperature sensor to substantially approach a same temperature as the cooling fluid when the first portion of the heat-conductive probe is immersed in the cooling fluid or disposed in direct physical contact with the fluid tank.

17. The method of claim 14, wherein the first portion of the temperature indicator comprises a thermochromic coating and the method comprises determining one of:
a presence of ice in the cooling fluid based on the thermochromic coating displaying the first color; or
an absence of ice in the cooling fluid based on the thermochromic coating displaying the second color.

18. The method of claim 17, wherein the determining is carried out by visual inspection of the thermochromic coating displaying either the first color or the second color through a lens covering the temperature indicator and configured to thermally insulate the temperature indicator from an ambient environment outside the cold therapy device.

19. The method of claim 17, comprising causing a temperature of the thermochromic coating to substantially approach a same temperature as the cooling fluid when the first portion of the heat-conductive probe is immersed in the cooling fluid.

20. A method of manufacturing a cold therapy device, the method comprising:
forming a fluid tank configured to hold a cooling fluid;
forming a lid configured to seal an opening in the fluid tank;
disposing at least a first portion of a temperature indicator on the lid or a side of the cold therapy device, the first portion of the temperature indicator configured to display one of a first color, intensity and/or pattern of light or a second color, intensity and/or pattern of light based on a temperature of the cooling fluid; and
disposing a second portion of a heat-conductive probe in physical contact with the temperature indicator, wherein a first portion of the heat-conductive probe is configured to be immersed in the cooling fluid or disposed in direct physical contact with the fluid tank and the second portion of the heat-conductive probe is configured to conduct heat between the first portion of the heat-conductive probe and the temperature indicator.

21. The method of claim 20, wherein the first portion of the temperature indicator comprises at least one light guide disposed at the side of the cold therapy device and configured to provide the first or second color, intensity and/or pattern of light externally of the cold therapy device, the method further comprising:
mounting a circuit board on or adjacent to the fluid tank, the circuit board forming a part of the temperature indicator and comprising:
a temperature sensor configured to generate an electrical signal indicative of the temperature of the cooling fluid,
one or more light emitting elements configured to generate the first or second color, intensity and/or pattern of light, and
circuitry configured to control the one or more light emitting elements based on the electrical signal.

22. The method of claim 20, wherein the first portion of the heat-conductive probe has a first diameter and the second portion of the heat-conductive probe has a second diameter smaller than the first diameter.

23. The method of claim 20, wherein the second portion of the heat-conductive probe is at least partially physically disposed within a recess within a portion of the temperature indicator.

24. The method of claim 20, wherein the first portion of the temperature indicator comprises a substantially dome-shaped thermochromic coating disposed on the lid, the thermochromic coating comprising a convex curvature that bows outward from the cold therapy device.

25. The method of claim 24, comprising disposing a lens over the temperature indicator, the lens configured to thermally insulate the temperature indicator from an ambient environment outside the cold therapy device.
